SEQUENCE LISTING (1 sequence)

SEQ ID NO: 1 - protein, 10 aa, Artificial Sequence
  CY101
LYDVAGSDKY

(19) United States
(12) Patent Application Publication
(10) Pub. No.: US 2022/0143152 A1
(43) Pub. Date: May 12, 2022

(54) USE OF PEPTIDE DERIVED FROM H-REV107

(71) Applicant: GJMEDI INC., Busan (KR)

(72) Inventors: Se Bok JANG, Busan (KR); MiSuk JEONG, Busan (KR); Chang Woo HAN, Busan (KR)

(73) Assignee: GJMEDI INC., Busan (KR)

(21) Appl. No.: 17/435,166

(22) PCT Filed: Feb. 28, 2020
(86) PCT No.: PCT/KR2020/002859

§ 371 Date: Aug. 31, 2021

(30) Foreign Application Priority Data
Apr. 4, 2019 (KR) 10-2019-0039602

(51) Int. Cl. A61K 38/46; A61K 9/00; A61K 47/46; A61K 47/26; A61K 9/48; A61K 9/16; A61K 9/20; A61P 35/00; A23L 33/00; A23L 33/18

(52) U.S. Cl. CPC A61K 38/465; A23V 2002/00; A61K 47/46; A61K 47/26; A61K 9/0019; A61K 9/4825; A61K 9/4858; A61K 9/4866; A61K 9/1611; A61K 9/1623; A61K 9/2018; A61K 9/2013; A61K 9/2059; A61P 35/00; A23L 33/40; A23L 33/18; A61K 9/0056

(57) ABSTRACT

The present invention relates to a novel use of a peptide derived from H-REV107 and, more particularly, to a composition for preventing ameliorating or treating cancer and an anticancer adjuvant composition comprising the peptide. The peptide derived from H-REV107 according to the present invention not only forms a complex with mutant KRAS, but also has an effect of inhibiting cancer through blocking an activity of RAS, and thus can be used in various ways in the fields of cancer prevention and treatment.

### TECHNICAL FIELD

The present invention relates to a novel use of a peptide derived from H-REV107, more particularly, to a composition for preventing, improving or treating cancer and an anticancer adjuvant composition, each of which includes the peptide described above.

### BACKGROUND ART

RAS is a primary oncogene mutated in human cancer, and a RAS protein is encoded by a gene of HRAS (Harvey-RAS), KRAS (Kirsten-RAS) or NRAS (neuroblastoma-RAS). The H-, K- and N-RAS proteins are small GTPases that act as signal transduction regulators involved in various processes such as cell differentiation, cell-cell adhesion, growth and death. The small GTPase RAS protein acts as a “molecular switch” that reciprocates between inactive and active states, and may be activated by conversion of GTP, which is promoted by guanine nucleotide exchange factors (GANs).

Activated mutations of RAS are found in specific human cancers. About 9-30% of human tumors have KRAS (86%), NRAS (11%) and HRAS (3%) activated mutations. Thereamong, KRAS was found in human cancers such as pancreatic cancer (90%), colon cancer (40%) and non-small cell lung cancer (20%), and the mutated cancer gene becomes a target of drug design. Cancers containing RAS mutants are aggressive and respond very little to standard treatments. In other words, since drug affinity for mutant KRAS is very low, it is difficult to directly target this oncogene, and drugs directly targeting the mutant RAS gene have not been designed. Molecules inhibiting mutant RAS developed so far have been reported to indirectly target RAS functional interactions without binding to RAS.

On the other hand, the most common mutant KRAS types are G12C, G12D and G12V, which account for 83% of all mutant KRAS. Thereamong, patients having ovarian carcinoma with the KRAS G12V mutant showed a shorter overall rate of survival than those without the above mutant. For this reason, selective targeting of the KRAS G12V mutant has emerged as a top priority in ovarian cancer treatment.

### DISCLOSURE

### Technical Problem

The present inventors have developed an H-REV107-derived peptide that directly targets the mutant KRAS while studying a method for directly targeting the RAS mutant, thereby completing the present invention.

Accordingly, an object of the present invention is to provide an anticancer peptide represented by an amino acid sequence of SEQ ID NO: 1.

Another object of the present invention is to provide a composition for preventing, improving or treating cancer, which includes the anticancer peptide represented by the amino acid sequence of SEQ ID NO: 1.

Another object of the present invention is to provide a cancer treatment method, which includes: treating an individual having cancer with an anticancer peptide represented by the amino acid sequence of SEQ ID NO: 1.

### Technical Solution

In order to achieve the above objects, the present invention provides an anticancer peptide represented by an amino acid sequence of SEQ ID NO: 1.

Further, the present invention provides a pharmaceutical composition for preventing or treating cancer, which includes the anticancer peptide represented by the amino acid sequence of SEQ ID NO: 1.

Further, the present invention provides a food composition for preventing or improving cancer, which includes the anticancer peptide represented by the amino acid sequence of SEQ ID NO: 1.

Further, the present invention provides an anticancer adjuvant composition which includes the anticancer peptide represented by the amino acid sequence of SEQ ID NO: 1.

In addition, the present invention provides a cancer treatment method, which includes: treating an individual having cancer with the anticancer peptide represented by the amino acid sequence of SEQ ID NO: 1.

### Advantageous Effects

The H-REV107-derived peptide according to the present invention not only forms a complex with mutant KRAS, but also has effects of inhibiting cancer through blocking activity of RAS, thereby being useable in various fields of cancer prevention and treatment.

### DESCRIPTION OF DRAWINGS

FIG. 1 is a diagram illustrating a complex crystal structure of an anticancer peptide CY101 and a mutant KRASG12V according to the present invention and an electron density map of the anticancer peptide CY101.

FIG. 2 illustrates results of measuring apparent dissociation constants of the anticancer peptide CY101 and mutant KRAS proteins G12D and G12V complex according to the present invention.

FIG. 3 illustrates results of confirming mutant KRAS binding inhibitory activity of the anticancer peptide CY101 according to the present invention through GTB binding assay.

FIG. 4 illustrates results of confirming a degree of cancer inhibitory activity of the anticancer peptide CY101 according to the present invention in different cancer cell-lines.

### DETAILED DESCRIPTION OF PREFERRED EMBODIMENTS OF INVENTION

Hereinafter, the present invention will be described in detail.

According to an aspect of the present invention, the present invention provides an anticancer peptide represented by an amino acid sequence of SEQ ID NO: 1.

In the present invention, “peptide” refers to a linear molecule formed by binding amino acid residues to each other through peptide bonds. The peptide may be prepared by a chemical synthesis method known in the art, and is preferably prepared according to a solid phase synthesis technique, but is not limited thereto.

In an embodiment of the present invention, the anticancer peptide is derived from H-REV107, which is a growth inhibitory RAS target gene capable of inhibiting non-adherent proliferation in vivo, and is preferably represented by the amino acid sequence of SEQ ID NO: 1. The anticancer peptide is composed of a short length of 10 amino acids, has advantages of easy mass production and commercialization, and exhibits remarkably high binding affinity for mutant KRAS to thus have specificity in selection of sequences.

The anticancer peptide according to the present invention may include functional equivalents and salts thereof. The “functional equivalent” refers to a peptide having at least 80% or more, preferably 90%, more preferably 95% or more sequence homology (i.e., identity) with the peptide of SEQ ID NO: 1 as a result of addition, substitution or deletion of amino acids. For example, those having sequence homology of “80%, 81%, 82%, 83%, 84%, 85%, 86%, 87%, 88%, 89%, 90%, 91%, 92%, 93%, 94%, 95%, 96%, 97%, 98%, 99% and 100%” are included and refer to peptides with substantially the same physiological activity as the peptide of SEQ ID NO: 1. In the present specification, sequence homology and homogeneity are defined as the percentage of amino acid residues in a candidate sequence relative to the amino acid sequence of SEQ ID NO: 1 after aligning the amino acid sequence of SEQ ID NO: 1 with the candidate sequence while introducing gaps. If necessary, conservative substitution is not considered as a part of sequence homogeneity in order to obtain the maximum percentage sequence identity. The N-terminus, C-terminus or internal extension, deletion or insertion of the amino acid sequence of SEQ ID NO: 1 is not interpreted as a sequence affecting sequence homogeneity or homology.

Further, the sequence identity may be determined by a general standard method used to compare similar portions of the amino acid sequences of two polypeptides. Computer programs such as BLAST or FASTA align the two polypeptides so that each amino acid is optimally matched (along the full length of one or two sequences, or along the predicted portions of one or two sequences). The program provides a default opening penalty and a default gap penalty, and a scoring matrix such as PAM250 (Standard Scoring Matrix; Dayhoff et al., Atlas of Protein Sequence and Structure, vol 5, supp. 3, 1978), which can be used in conjunction with a computer program). For example, percentage homogeneity may be calculated as follows: after multiplying the total number of identical matches by 100, the resulting value is divided by the sum of the length of the longer sequence within the matched span and the number of gaps introduced into the longer sequence in order to align these two sequences.

In the above, “substantially homogenous physiological activity” refers to anticancer activity. The range of the “functional equivalent” according to the present invention may include derivatives in which some chemical structures of the peptide are modified while maintaining the basic skeleton of the peptide of SEQ ID NO: 1 and anticancer activity. This may include, for example, structural changes to alter the stability, storage, volatility or solubility of the peptide.

According to an embodiment of the present invention, the anticancer peptide preferably targets at least one selected from the group consisting of mutant KRAS G12V, G12D, G12C, G13D and Q61H, more preferably mutant KRAS G12V, but is not limited thereto.

According to the present invention, “cancer” is a generic term for diseases caused by cells having aggressive properties wherein cells divide and grow while ignoring normal growth limitation, invasive properties wherein cells penetrate into surrounding tissues, and metastatic properties wherein cells spread to other parts of the body. The cancer is preferably one or more selected from the group consisting of gastric cancer, breast cancer, lung cancer, liver cancer, blood cancer, bone cancer, pancreatic cancer, skin cancer, head or neck cancer, cutaneous or intraocular melanoma, uterine sarcoma, ovarian cancer, rectal cancer, anal cancer, colon cancer, fallopian tube carcinoma, endometrial carcinoma, cervical cancer, small intestine cancer, endocrine cancer, thyroid cancer, parathyroid cancer, adrenal cancer, soft tissue tumor, urethral cancer, prostate cancer, bronchogenic cancer and bone marrow tumor, but is not limited thereto.

According to an embodiment of the present invention, the anticancer peptide may not only form a complex with the mutant KRAS but also inhibit formation of a complex with H-REV107 by blocking activity of RAS, that is, has an effect of inhibiting cancer, thereby being useable in various ways in the field of prevention, improvement or treatment.

According to another aspect of the present invention, the present invention may provide a pharmaceutical composition for preventing or treating cancer, which includes an anticancer peptide represented by an amino acid sequence of SEQ ID NO: 1.

When the composition of the present invention is used as a pharmaceutical composition, the pharmaceutical composition of the present invention may be formulated and used in various forms according to any conventional method. For example, the composition may be formulated in oral dosage forms such as powders, granules, tablets, capsules, suspensions, emulsions, syrups, etc., and may be formulated in the form of external preparations, suppositories and sterile injectable solutions.

The composition of the present invention may contain one or more known active ingredients having prophylactic or therapeutic effects on cancer together with an anticancer peptide.

The composition of the present invention may further include a pharmaceutically acceptable additive, wherein the pharmaceutically acceptable additive includes starch, gelatinized starch, microcrystalline cellulose, lactose, povidone, colloidal silicon dioxide, calcium hydrogen phosphate, mannitol, syrup, arabic rubber, pregelatinized starch, corn starch, powdered cellulose, hydroxypropyl cellulose, Opadry, sodium starch glycolate, carnauba wax, synthetic aluminum silicate, stearic acid, magnesium stearate, aluminum stearate, calcium stearate, white sugar or the like may be used. The pharmaceutically acceptable additive according to the present invention is preferably included in an amount of 0.1 to 90 parts by weight (“wt. parts”) based on the composition, but is not limited thereto.

The composition of the present invention may be administered in various oral or parenteral dosage forms at the time of actual clinical administration. When formulated, diluents or excipients such as fillers, extenders, binders, wetting agents, disintegrants, surfactants, etc., may be used. It is preferable to use the materials disclosed in literature (Remington's Pharmaceutical Science, latest edition, Mack Publishing Company, Easton Pa.) as a suitable formulation known in the art.

The solid preparations for oral administration may include tablets, pills, powders, granules, capsules, and the like, and such solid preparations may be prepared by mixing the composition with at least one excipient, such as starch, calcium carbonate, sucrose, lactose, gelatin, etc. In addition to simple excipients, lubricants such as magnesium stearate and talc may also be used. Further, liquid preparations for oral administration may include suspensions, solvents, emulsions, syrups, and the like, and may further include various excipients other than water and liquid paraffin as commonly used simple diluents, for example, wetting agents, sweeteners, fragrances, preservatives, etc.

Formulations for parenteral administration may include sterilized aqueous solutions, non-aqueous solutions, suspensions, emulsions, lyophilized formulations, and suppositories. The non-aqueous solvents or suspensions may include propylene glycol, polyethylene glycol, vegetable oil such as olive oil, injectable ester such as ethyl oleate, etc. Meanwhile, a basic material of the suppository agent may include Witepsol, macrogol, tween 61, cacao butter, laurin butter and glycerol-gelatin.

An administration amount (“dosage”) of the pharmaceutical composition of the present invention may vary depending on the formulation method, administration method, administration time and/or route of administration of the pharmaceutical composition, and type and degree of reaction to be achieved by administration of the pharmaceutical composition, type of subject to be administered, age, weight, general health condition, symptoms or severity of disease, sex, diet, excretion, various factors including components of drugs and/or other compositions used simultaneously or separately for the subject, and similar factors well known in the medical field. Further, persons of ordinary skill in the art (“those skilled in the art”) may easily determine and prescribe an effective dosage for desired treatment.

The administration route and the administration method of the pharmaceutical composition according to the present invention may each be independent, and are not particularly limited in terms of manners for the same. Further, any route and method of administration may be adopted as long as the pharmaceutical composition can reach the desired site.

The pharmaceutical composition of the present invention may be used alone or in combination with surgery, radiation therapy, hormone therapy, chemotherapy, and use of a biological response modifier for prevention or treatment of cancer.

According to another aspect of the present invention, the present invention may provide a food composition for preventing or improving cancer, which includes the anticancer peptide represented by the amino acid sequence of SEQ ID NO: 1.

When the composition of the present invention is used as a food composition, the food composition of the present invention refers to a food having effects of preventing or improving cancer and diseases caused by cancer, and should be harmless to the human body even when taken for a long time.

There is no particular limitation on the type of food. Examples of foods to which the above substances can be added may include meat, sausage, bread, chocolate, candy, snacks, confectionery, pizza, ramen, other noodles, gum, dairy products including ice cream, soups, beverages, tea, drinks, alcoholic beverages and vitamin complexes, and all health foods in the usual sense are included.

According to an embodiment of the present invention, the food composition of the present invention may be a food additive. The food additive may be added to the anticancer peptide as it is or may be used along with other foods or food ingredients, and may be appropriately used according to any conventional method. A mixing amount of active ingredients may be appropriately determined according to the purpose of use (prevention, health or therapeutic treatment).

According to an embodiment of the present invention, the food composition of the present invention may be a health beverage composition. Like typical beverages, the health beverage composition of the present invention may include various flavoring agents or natural carbohydrates as additional components, in addition to the anticancer peptide. The natural carbohydrate described above may include, for example, monosaccharides such as glucose or fructose, disaccharides such as maltose, sucrose, etc., natural sweeteners such as dextrin, cyclodextrin, etc., as well as synthetic sweeteners such as saccharine, aspartame, etc. A ratio of the natural carbohydrates may range from about 0.01 to 10 g, preferably about 0.01 to 0.1 g per 100 ml of the composition of the present invention.

In addition to the above, the composition of the present invention may include various nutrients, vitamins, electrolytes, flavoring agents, colorants, pectic acid and salts thereof, alginic acid and salts thereof, organic acids, protective colloidal thickeners, pH adjusters, stabilizers, preservatives, glycerin, alcohols, carbonation agents used in carbonated beverages. In addition, the composition of the present invention may include fruit flesh for manufacturing natural fruit juice, fruit juice beverage and vegetable beverage. These components may be used independently or in combination thereof. Proportions of these additives are not very significant, but may be generally selected from 0.01 to 0.1 wt. parts per 100 wt. parts of the composition according to the present invention.

Further, the present invention may provide an anticancer adjuvant composition including then anticancer peptide represented by the amino acid sequence of SEQ ID NO: 1.

According to the present invention, “anticancer adjuvant” refers to an agent that can be additionally used to enhance effects of cancer therapeutics generally used in the art, and using the adjuvant according to the present invention may promote effects of the cancer therapeutics or anticancer treatment effects.

The anticancer adjuvant composition of the present invention may be in the form of a pharmaceutical composition or a food composition, and more specifically, may be an anticancer pharmaceutical adjuvant or an anticancer food supplement.

According to another aspect of the present invention, the present invention may provide a cancer treatment method, including: treating an individual suffering from cancer with the anticancer peptide represented by the amino acid sequence of SEQ ID NO: 1.

Hereinafter, the present invention will be described in more detail by way of examples. These examples are for illustrative purposes only, and it will be apparent to those of ordinary skill in the art that the scope of the present invention is not construed as being limited by these examples.

### Example 1. Anticancer Peptide Design and Production

Conventionally developed cancer treatment agents have very low affinity for mutant KRAS (Kirsten-RAS) and, therefore, it is difficult to directly target this oncogene. Further, since the mechanism related to affinity of the mutant KRAS has not been identified, it is difficult to determine its structure. Therefore, studies were conducted on a tumorigenic mutant KRAS and H-REV107 (HRAS-like suppressor 3) complex. As a result, interaction between the mutant KRAS and H-REV107 complex was subjected to molecular modeling, and an anticancer peptide was designed based on results thereof. The designed anticancer peptide was derived from H-REV107, was represented by an amino acid sequence of SEQ ID NO: 1, and was named “CY101”. The anticancer peptide CY101 was produced using Fomc solid-phase peptide synthesis (SPPS) and purified by reverse-phase high performance liquid chromatography (RP-HPLC) having a purity of 95% or more. The purified anticancer peptide CY101 was identified using liquid chromatography/mass spectrometry (LC-MS).

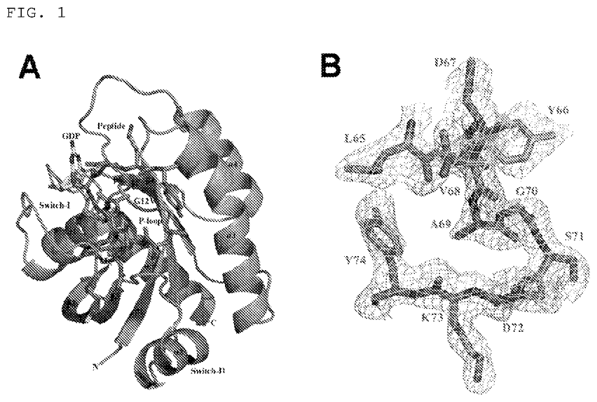

The interaction and crystal structure of the prepared anticancer peptide CY101 and the carcinogenic mutant KRAS G12V were determined by X-ray crystallography. More specifically, crystals of the KRAS G12V-CY101 complex were grown in a solution containing polystyrene glycol 3,350, potassium nitrate (pH 6.8) and acetonitrile. Crystals of the KRAS G12V-CY101 complex were immersed in a cryogenic protective solution containing glycerol, followed by freezing the crystals in liquid nitrogen at 100 K or less for data collection. The X-ray diffraction data of the KRAS G12V-CY101 composite was collected on beam line 7A of Pohang Accelerator Research Institute. The collected diffraction data was processed with HKL-2000 software, and the structure was analyzed with CCP4 by molecular substitution. The final model was subdivided and created using COOT and PHENIX, and all structural figures and electron density maps were created using PyMOL program. The crystal structure of the KRAS G12V-CY101 complex and the electron density map of CY101 are shown in FIG. 1.

As shown in FIG. 1, it was confirmed that the anticancer peptide CY101 forms a complex with the oncogenic mutant KRAS G12V. The complex was determined at a resolution of 2.3 Å (FIG. 1a). It was confirmed that the electron density map of the anticancer peptide CY101 was outlined as 1σ (gray).

### Example 2. Measurement of Apparent Dissociation Constant of Anticancer Peptide CY101 and Mutant KRAS Protein

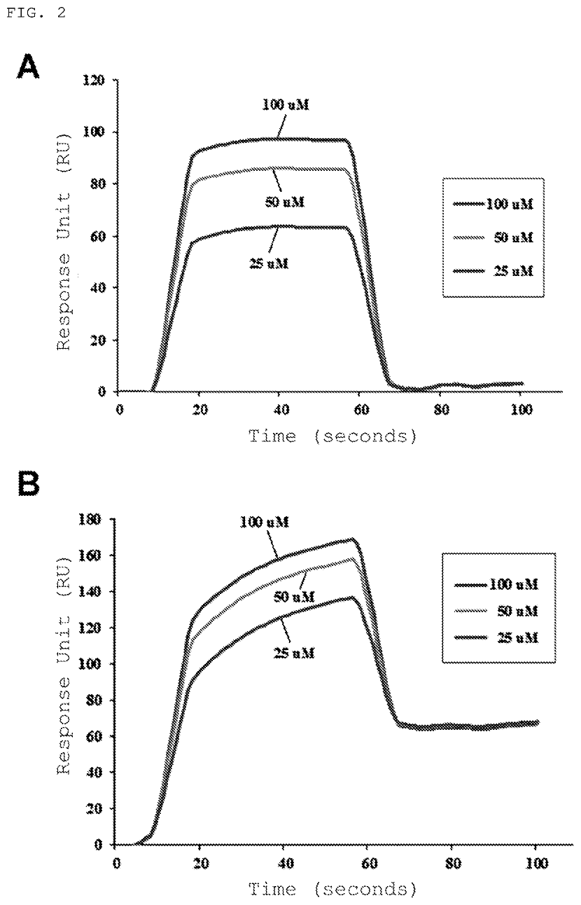

The apparent dissociation constant (K_{d}) between the anticancer peptide CY101 prepared in Example 1 and the mutant KRAS G12D and G12V proteins was measured using a Biacore T100 biosensor. Specifically, each mutant KRAS protein contained in 10 mM sodium acetate (pH 5.0) was attached to the CM5 sensor chip at a concentration corresponding to 2,300 response units (RU) by an amine attachment method. Kinetic parameters from the cells contained in the sensor chip, to which the mutant KRAS protein is attached, to the other cells contained in the induced chip were simultaneously measured. Two rheological paths were used to determine motion parameters. For kinetic measurement at room temperature, samples were prepared by diluting CY101 in HBS buffer (150 mM NaCl, 3 mM EDTA, 10 mM HEPES and 0.005% surfactant P20, pH 7.4) until the respective concentrations reached 25, 50 and 100 μM, respectively. After injecting each sample into the sensor chip at a rate of 10 μl/min, sodium hydroxide (50 mM) as a fixed ligand was injected and regenerated. The results of measuring the apparent dissociation constants of the anticancer peptide CY101 and the mutant KRAS protein G12D and G12V complex are shown in FIG. 2.

As shown in FIG. 2, it was confirmed that the anticancer peptide CY101 forms a complex with the mutant KRAS G12D (FIG. 2a) and G12V (FIG. 2b).

### Example 3. Confirmation of Mutant KRAS Binding Inhibitory Activity of Anticancer Peptide CY101 Through GTP Binding Assay

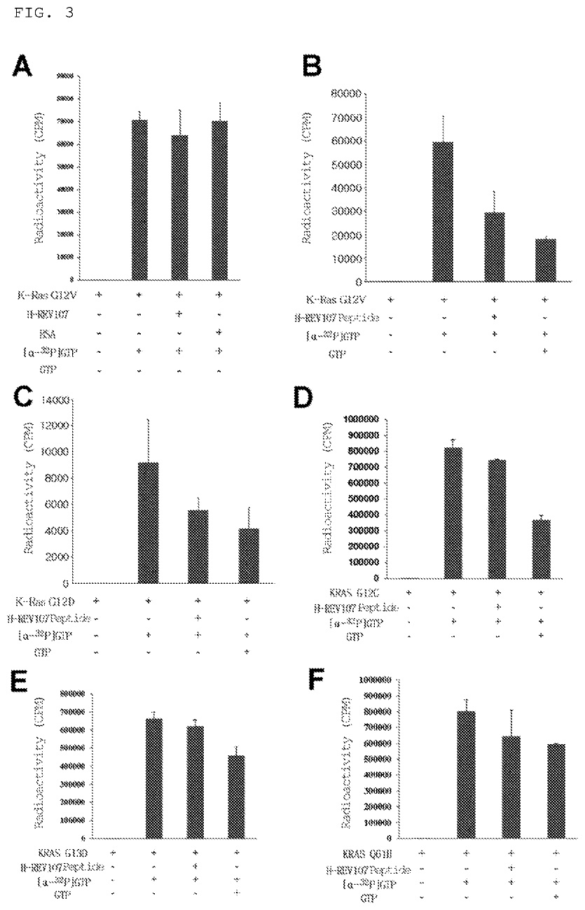

It was investigated whether the anticancer peptide CY101 prepared in Example 1 exhibits inhibitory activity against a variety of mutant KRAS. Specifically, each of His-labeled mutant KRAS proteins (G12V, G12D, G12C, G13D and Q61H) and the anticancer peptide CY101 were mixed in a molar ratio of 1:1 to prepare a mixture thereof. The prepared mixture was incubated with a binding solution (50 mM Hepes (pH 7.5), 100 mM NaCl, 2 mM MgCl₂, 1 mM EDTA and 1 mM DTT). The culture was passed through Ni-NTA resin and then bound at 4° C. Also, a CY101-mutant KRAS protein complex was incubated with [α^{-32P}] GTP (2,500 cpm/pmol) and GTP at 30° C. To terminate binding, a washing buffer (20 mM Tris-HCl (pH 7.4), 100 mM NaCl and 2 mM MgCl₂) was added. After washing, the bound protein was eluted using 200 mM imidazole. In this example, bovine serum albumin was used as a negative control. Radioactive GTP bound to the protein was quantified by a liquid scintillation counter, and the results thereof are shown in FIG. 3.

As shown in FIG. 3a, it was confirmed that the mutant KRAS G12V and BSA added group had an increase in radioactive GTP binding activity. However, in the presence of H-REV107 protein, the radioactive GTP binding activity of the mutant KRAS G12V was somewhat reduced.

Further, as shown in FIGS. 3b to 3f, it was confirmed that, when the anticancer peptide CY101 is present, radioactive GTP binding to the mutant KRAS protein is significantly reduced. Specifically, after addition of the anticancer peptide CY101, GTP binding to the mutant KRAS proteins G12V, G12D and G12C was reduced by 50, 40 and 10%, respectively, compared to the GTP binding activity for the KRAS mutant. On the other hand, GTP binding to Q61H and G13D were decreased by 20% and 7%, respectively. The above results indicate that the anticancer peptide CY101 inhibits the interaction between the mutant KRAS proteins G12V, G12D, G12C, G13D and Q61H, respectively, and the GTP, thereby blocking RAS activation function.

### Example 4. Confirmation of Cancer Inhibitory Activity of Anticancer Peptide CY101 in Various Cancer Cell-Lines

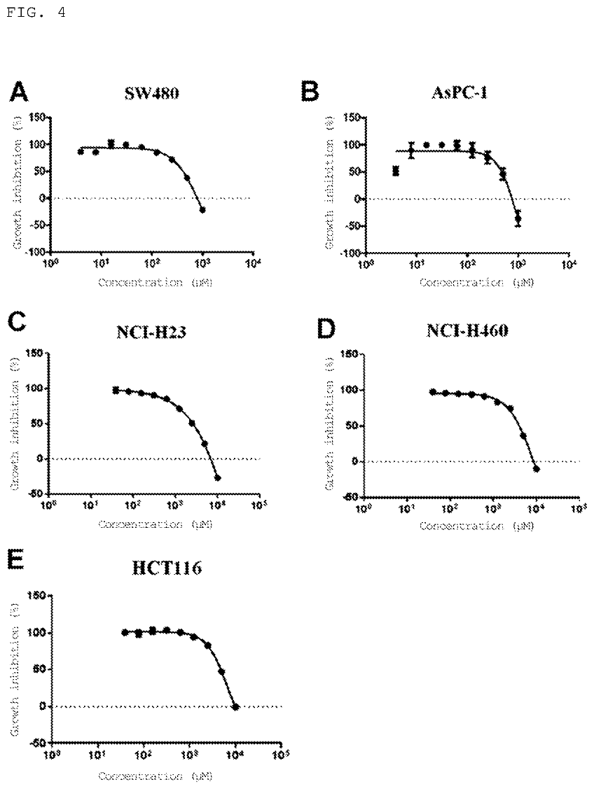

A cancer cell-line containing the mutant KRAS was used to assess cancer inhibitory activity of the anticancer peptide CY101. Cancer cell-lines used in this example were SW480 (colon cancer), AsPC-1 (pancreatic cancer), NCI-H23 (lung cancer), NCI-H460 (lung cancer) and HCT116 (colon cancer) cell-lines. Specifically, for HCT116 and NCI-H460 cell-lines, 100 μl of a culture medium containing 2×10³ cells per well; for SW480 and NCI-H23 cell-lines, 100 μl of a culture medium containing 5×10³ cells per well; and, for AsPC-1 cell-line, 100 μl of a culture solution containing 1×10⁴ cells per well, respectively, were seeded. The seeded cells were cultured for 24 hours for adhesion to the well, and the medium was changed. The cells were cultured for 72 hours after treatment with the anticancer peptide CY101. After completion of the culture, a cell counting kit-8 (CCK-8) solution (10 μl/well) was added, and the cells were further cultured for 4 hours in a CO₂ incubator maintained at 37° C. Absorbance (OD) of each well at 450 nm was measured using a microplate reader, and the results are shown in FIG. 4.

As shown in FIG. 4, it was confirmed that a GI₅₀ value (Half maximal growth inhibition concentration) of the anticancer peptide CY101 was 358 and 417 μM for SW480 and AsPC-1 cell lines, respectively. Further, it was confirmed that the anticancer peptide CY101 against NCI-H23, NCI-H460 and HCT-116 cell-lines, respectively, had a GI₅₀ value in the millimolar (mM) range. The above results mean that the anticancer peptide CY101 exhibits excellent anticancer activity even at a low concentration.

In summary, the present inventors have developed an anticancer peptide CY101, and confirmed that the anticancer peptide CY101 not only forms a complex with the mutant KRAS, but also blocks the activity of RAS. This means that the anticancer peptide CY101 has cancer inhibitory activity, and may be used in various ways in the field of cancer prevention and treatment.

Hereinafter, the present invention will be described in more detail by means of the following preparative examples. These preparative examples are for illustrative purposes only, and the scope of the present invention is not construed as being limited thereby.

### Preparative Example 1. Preparation of Pharmaceutical Composition for Preventing or Treating Cancer

1-1. Preparation of Powder

1 mg of anticancer peptide represented by the amino acid sequence of SEQ ID NO: 1

100 mg lactose

10 mg of talc

The above ingredients were mixed and filled in an airtight cloth to prepare a powder.

1-2. Preparation of Tablets

1 mg of anticancer peptide represented by the amino acid sequence of SEQ ID NO: 1

100 mg of corn starch

100 mg of lactose

2 mg of magnesium stearate

After mixing the above ingredients, tablets are prepared by tableting according to a conventional tablet preparation method.

1-3. Preparation of Capsules

1 mg of anticancer peptide represented by the amino acid sequence of SEQ ID NO: 1

3 mg of crystalline cellulose

14.8 mg of lactose

0.2 mg of magnesium stearate

According to a conventional capsule preparation method, the above ingredients are mixed and filled into gelatin capsules to prepare a capsule.

1-4. Preparation of Injections

1 mg of anticancer peptide represented by the amino acid sequence of SEQ ID NO: 1

180 mg of mannitol

2974 mg of sterile distilled water for injection

0.26 mg of Na₂HPO₄.2H₂O 26 mg

The injection is prepared with the above ingredients per ampoule (2 ml) according to a conventional injection preparation method.

1-5. Preparation of Liquid

1 mg of anticancer peptide represented by the amino acid sequence of SEQ ID NO: 1

10 g of isomerized sugar

5 g of mannitol

Appropriate amount of purified water

After adding and dissolving each component in purified water according to the usual preparation method of liquid and further adding a proper amount of lemon zest, the above ingredients are mixed, followed by adding purified water to adjust a total amount to 100 ml water, and then, the mixture is filled in a brown bottle for sterilization, thereby preparing a solution.

As described above, the present invention has been concretely described in detail and, for those skilled in the art, it is obvious that this specified description is proposed as only a preferred embodiment, and the scope of the present invention is not limited thereby. Accordingly, it will be considered that the substantial scope of the present invention is defined by the appended claims and their equivalents.

1. An anticancer peptide represented by an amino acid sequence of SEQ ID NO: 1.

2. The anticancer peptide according to claim 1, wherein the anticancer peptide is derived from H-REV107.

3. The anticancer peptide according to claim 1, wherein the anticancer peptide inhibits formation of a complex of a mutant KRAS (Kirsten-RAS) and H-REV107 (HRAS-like suppressor 3).

4. The anticancer peptide according to claim 3, wherein the anticancer peptide targets one or more selected from the group consisting of mutants KRASG12V, G12D, G12C, G13D and Q61H.

5. The anticancer peptide according to claim 1, wherein the cancer is one or more selected from the group consisting of gastric cancer, breast cancer, lung cancer, liver cancer, blood cancer, bone cancer, pancreatic cancer, skin cancer, head or neck cancer, cutaneous or intraocular melanoma, uterine sarcoma, ovarian cancer, rectal cancer, anal cancer, colon cancer, fallopian tube carcinoma, endometrial carcinoma, cervical cancer, small intestine cancer, endocrine cancer, thyroid cancer, parathyroid cancer, adrenal cancer, soft tissue tumor, urethral cancer, prostate cancer, bronchogenic cancer and bone marrow tumor.

6. A pharmaceutical composition for prevention or treatment of cancer, comprising the anticancer peptide according to any one of claims 1 to 5.

7. A food composition for prevention or improvement of cancer, comprising the anticancer peptide according to any one of claims 1 to 5.

8. An anticancer adjuvant composition, comprising the anticancer peptide according to any one of claims 1 to 5.

9. A cancer treatment method, comprising: treating an individual having cancer with an anticancer peptide represented by an amino acid sequence of SEQ ID NO: 1.
